(19) United States
(12) Patent Application Publication
(10) Pub. No.: US 2024/0099694 A1
(43) Pub. Date: Mar. 28, 2024

(54) ULTRASOUND PROBE AND ULTRASOUND DIAGNOSTIC APPARATUS

(71) Applicant: FUJIFILM Corporation, Tokyo (JP)

(72) Inventor: Atsushi OSAWA, Ashigarakami-gun (JP)

(73) Assignee: FUJIFILM Corporation, Tokyo (JP)

(21) Appl. No.: 18/472,163

(22) Filed: Sep. 21, 2023

(30) Foreign Application Priority Data
Sep. 22, 2022 (JP) 2022-151552

(51) Int. Cl. A61B 8/00; G10K 11/30

(52) U.S. Cl. CPC A61B 8/4444; A61B 8/4494; A61B 8/546; G10K 11/30

(57) ABSTRACT

An ultrasound probe includes: a vibrator having a first surface which is located on a side on which the vibrator transmits and receives an ultrasound wave and a second surface opposite to the first surface; a support portion that supports the second surface; an acoustic lens that is disposed on a side opposite to a support portion side with respect to the vibrator; an acoustic matching portion that is disposed between the vibrator and the acoustic lens; and a connection portion that connects the support portion and at least a third acoustic matching layer of the acoustic matching portion to each other, in which the support portion, the connection portion, and the third acoustic matching layer have the same thermal conductivity and the same acoustic impedance.

### CROSS-REFERENCE TO RELATED APPLICATIONS

This application is based upon and claims the benefit of priority from prior Japanese patent application No. 2022-151552, filed on Sep. 22, 2022, the entire contents of which are incorporated herein by reference.

### BACKGROUND OF THE INVENTION

### 1. Field of the Invention

The present invention relates to an ultrasound probe and an ultrasound diagnostic apparatus.

### 2. Description of the Related Art

JP2012-513714A describes an ultrasound converter comprising a matching body, a compensating body inserted between the matching body and a piezoelectric converter element, and an attenuation element, in which the attenuation element is configured to surround the matching body on a radiation side facing a fluid medium and to surround the matching body and the compensating body in a radial direction.

JP2004-329495A describes an ultrasound probe including a backing material in which a plurality of protruding portions are formed.

JP2015-181541A describes an ultrasound probe comprising: a backing material, a plurality of piezoelectric elements arranged on a surface of the backing material, a heat collection portion that has at least one heat conduction path extending in a thickness direction inside the backing material and of which a distal end portion is exposed from the surface of the backing material, which faces lower surfaces of the plurality of piezoelectric elements, that is made of a material having a thermal conductivity higher than a thermal conductivity of the backing material, and that absorbs heat from the plurality of piezoelectric elements, and a heat discharge portion that is connected to the heat collection portion and that discharges the heat absorbed by the heat collection portion to an outside.

### SUMMARY OF THE INVENTION

In an ultrasound diagnostic apparatus for a medical use, an ultrasound image is generated by transmitting an ultrasound beam from an ultrasound probe toward a subject under examination, receiving an ultrasound echo from the subject under examination through the ultrasound probe, and electrically processing a reception signal thereof. In recent years, with the advancement in performance of the ultrasound probe and the ultrasound diagnostic apparatus, various types of driving have been applied to a vibrator inside the ultrasound probe. As a result, there is a tendency for an increased amount of heat generation near a contact surface of the ultrasound probe with the subject under examination. Therefore, it is required to prevent the heat generated in the ultrasound probe from being transferred to the subject under examination. JP2012-513714A does not pertain to a technology that assumes an ultrasound diagnostic apparatus for a medical use.

An object of the present disclosure is to provide an ultrasound probe capable of restraining heat generated near a contact surface with a subject under examination from being transferred to the subject under examination, and an ultrasound diagnostic apparatus comprising the same.

According to one aspect of the present disclosure, there is provided an ultrasound probe comprising: at least one vibrator having a first surface which is located on a side on which the vibrator transmits and receives an ultrasound wave and a second surface opposite to the first surface; a support portion that supports the second surface; an acoustic lens that is disposed on a side opposite to a support portion side with respect to the vibrator; an acoustic matching portion that is disposed between the vibrator and the acoustic lens; and a connection portion that connects a first range and the support portion to each other, the first range being at least a partial range of the acoustic matching portion in a first direction from the acoustic lens toward the vibrator, in which the support portion, the connection portion, and the first range have the same thermal conductivity and the same acoustic impedance.

According to one aspect of the present disclosure, there is provided an ultrasound diagnostic apparatus comprising: the ultrasound probe described above.

According to the present disclosure, it is possible to provide an ultrasound probe capable of restraining heat generated near a contact surface with a subject under examination from being transferred to the subject under examination, and an ultrasound diagnostic apparatus comprising the same.

### BRIEF DESCRIPTION OF THE DRAWINGS

FIG. 1 is a perspective view partially showing a distal end part of an ultrasound probe 100 of one aspect according to the present disclosure.

FIG. 2 is a schematic view showing a cross-section of a unit U perpendicular to a left-right direction X.

FIG. 3 is a view showing a first modification example of the ultrasound probe 100 and is a schematic cross-sectional view corresponding to FIG. 2.

FIG. 4 is a view showing a second modification example of the ultrasound probe 100 and is a schematic cross-sectional view corresponding to FIG. 2.

### DESCRIPTION OF THE PREFERRED EMBODIMENTS

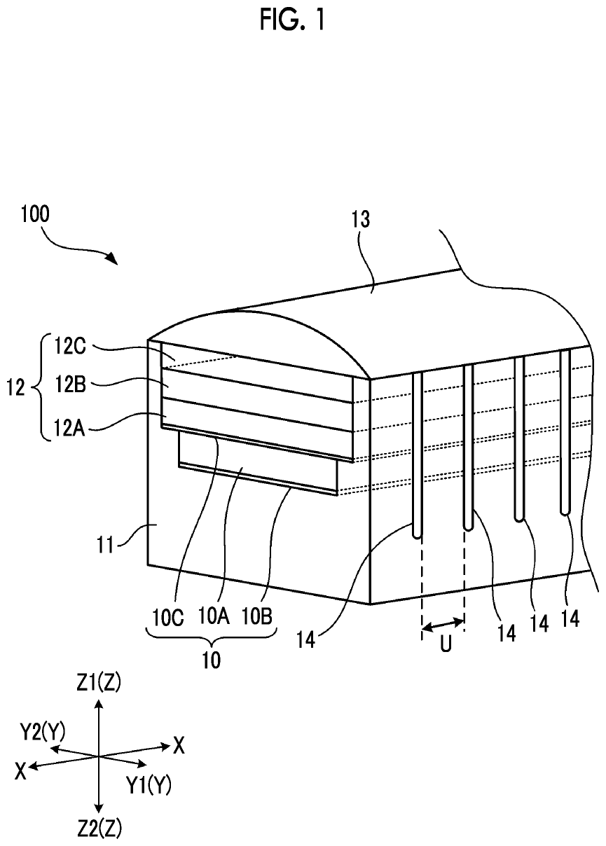

FIG. 1 is a perspective view partially showing a distal end part of an ultrasound probe 100 of one aspect according to the present disclosure. FIG. 1 shows three directions (a left-right direction X, a front-rear direction Y, and an up-down direction Z) that are orthogonal to each other as directions of the ultrasound probe 100. One of the up-down direction Z is referred to as an upward direction Z1, and a direction opposite to the upward direction Z1 is referred to as a downward direction Z2. One of the front-rear direction Y is referred to as a rearward direction Y1, and a direction opposite to the rearward direction Y1 is referred to as a forward direction Y2. The ultrasound probe 100 is used by bringing an upper-side end surface thereof into contact with a subject under examination. The left-right direction X and the front-rear direction Y are each a direction along a plane (one of the planes intersecting the up-down direction Z) perpendicular to the up-down direction Z. In the present specification, the up-down direction Z constitutes a first direction toward the vibrator 10 from the acoustic lens 13, which will be described below, the left-right direction X constitutes a second direction, and the front-rear direction Y constitutes a third direction intersecting the second direction.

The ultrasound probe 100 is an image generation device provided in the ultrasound diagnostic apparatus. The ultrasound diagnostic apparatus includes a device that generates and records an ultrasound image while bringing the ultrasound probe 100 into proximity with an outer surface of the subject under examination, a device that generates and records an ultrasound image while bringing the ultrasound probe 100 incorporated into an insertion part distal end of an endoscope into proximity with an organ of the subject under examination, or the like.

As shown in FIG. 1, the ultrasound probe 100 comprises a plurality of units U arranged in the left-right direction X, and an acoustic lens 13 provided on an upper side with respect to the plurality of units U and common to the plurality of units U. Adjacent units U are separated by a separation layer 14. The separation layer 14 is formed of an insulating resin material or the like. In the unit U, a width in the front-rear direction Y is greater than the width in the left-right direction X.

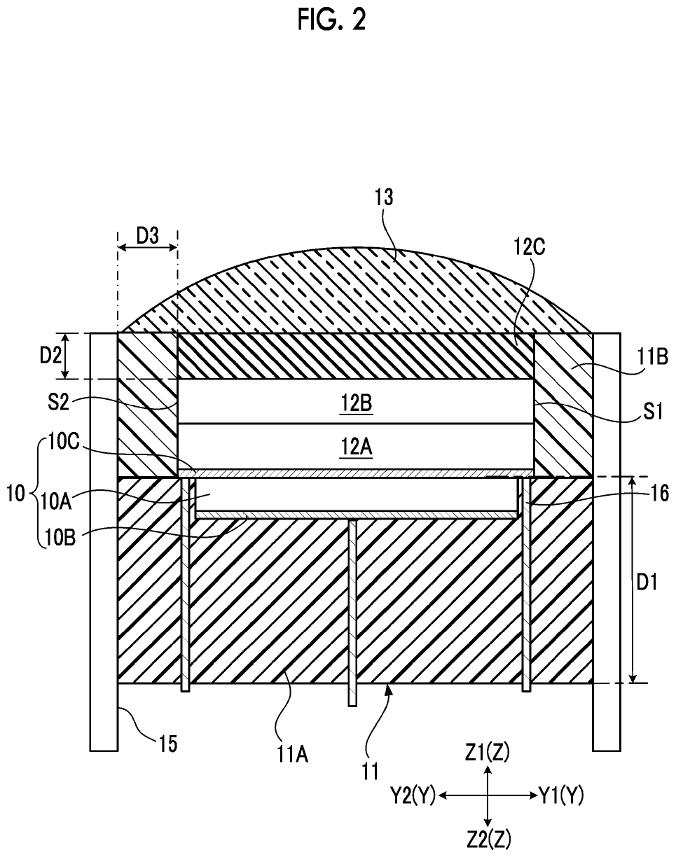

The unit U comprises the vibrator 10, an acoustic matching portion 12 disposed on the upper side with respect to the vibrator 10, and a functional part 11 provided around the vibrator 10 and the acoustic matching portion 12. Although not shown in FIG. 1, the acoustic lens 13, the plurality of units U, and the separation layer 14 are supported by a case 15 (see FIG. 2).

The vibrator 10 comprises a piezoelectric body 10A, a signal electrode 10B stuck to a lower-side surface of the piezoelectric body 10A, and a ground electrode 10C stuck to an upper-side surface of the piezoelectric body 10A. The piezoelectric body 10A generates an ultrasound wave in response to a voltage application and generates a reception voltage in a case where a reflected wave of the ultrasound wave is received. The piezoelectric body 10A is made of a piezoelectric ceramics, such as lead zirconate titanate (PZT), or a piezoelectric material, such as a polymer material, like polyvinylidene fluoride (PVDF) or the like. The piezoelectric body 10A may be composed of capacitive micro ultrasound transducers (CMUT), which are based on a semiconductor material, or the like.

The acoustic matching portion 12 is provided in order to efficiently transmit and receive ultrasound waves by matching acoustic impedances of the piezoelectric body 10A and the subject under examination. The acoustic matching portion 12 is preferably formed of a material having an acoustic impedance that is a value lower than the acoustic impedance of the piezoelectric body 10A and higher than the acoustic impedance of the subject under examination.

In the present embodiment, the acoustic matching portion 12 is formed by laminating a plurality of layers formed of such a material in the up-down direction Z. Specifically, the acoustic matching portion 12 comprises a first acoustic matching layer 12A stuck to an upper-side surface of the vibrator 10, a second acoustic matching layer 12B stuck to an upper-side surface of the first acoustic matching layer 12A, and a third acoustic matching layer 12C stuck to an upper-side surface of the second acoustic matching layer 12B. It is preferable that the acoustic matching portion 12 has a layered structure in which the acoustic impedance decreases in a stepwise manner from the vibrator 10 toward the subject under examination. The third acoustic matching layer 12C is made of an insulating material and is mainly made of, for example, a urethane resin, an epoxy resin, a silicone resin, or the like.

The acoustic lens 13 is provided in order to improve a resolving power by using refraction to focus the ultrasound beam. The acoustic lens 13 is generally composed of a convex type. The acoustic lens 13 is made of, for example, a silicone resin or plastic. The acoustic lens 13 is stuck to upper surfaces of the acoustic matching portions 12 in all the units U and an upper surface of the separation layer 14 between the units U with an adhesive or the like.

In the ultrasound probe 100, by applying a pulsed or continuous wave-like voltage between the ground electrodes 10C and the signal electrodes 10B of a plurality of the vibrators 10, each of the piezoelectric bodies 10A expands and contracts to generate a pulsed or continuous wave-like ultrasound wave. In a case where these ultrasound waves are incident on the subject under examination via the acoustic matching portion 12 and the acoustic lens 13, the ultrasound waves are combined with each other to form an ultrasound beam and propagate through the subject under examination. In a case where an ultrasound echo propagating through and reflected in the subject under examination is incident on each of the piezoelectric bodies 10A via the acoustic lens 13 and the acoustic matching portion 12, each of the piezoelectric bodies 10A is deformed, and in response to this deformation, a signal voltage is generated between the ground electrode 10C and the signal electrode 10B. The signal voltage generated in the plurality of vibrators 10 is extracted from between the ground electrode 10C and the signal electrode 10B of each of the vibrators 10 and received as a reception signal, and an ultrasound image is generated based on the reception signal.

FIG. 2 is a schematic view showing a cross-section of the unit U perpendicular to the left-right direction X. As shown in FIG. 2, the functional part 11 comprises a support portion 11A that supports a second surface (lower-side surface) opposite to a first surface (upper-side surface) which is located on a side on which the vibrator 10 transmits and receives ultrasound waves, and a connection portion 11B that connects the support portion 11A and at least a part of the acoustic matching portion 12 to each other. The support portion 11A supports a lower surface of the vibrator 10 by being in contact with a lower surface of the signal electrode 10B, front and rear end surfaces of the piezoelectric body 10A, and lower surfaces of both front and rear end parts of the ground electrode 10C. FIG. 2 shows a first end surface S1 and a second end surface S2 as both end surfaces of the acoustic matching portion 12 in the front-rear direction Y.

In the example of FIG. 2, the connection portion 11B connects the entire first end surface S1 and the entire second end surface S2 to the support portion 11A. More specifically, the connection portion 11B extends from upper surfaces of both front and rear end parts of the support portion 11A in the upward direction Z1 along the first end surface S1 and the second end surface S2, respectively, and is in contact with the first end surface S1 and the second end surface S2. The connection portion 11B is provided so as to fill a space between the case 15, and the first end surface S1 and the second end surface S2. In the present embodiment, the acoustic lens 13 also covers an upper surface of the connection portion 11B. That is, the connection portion 11B is in contact with the acoustic matching portion 12 and is also in contact with a lower surface of the acoustic lens 13.

The support portion 11A supports the plurality of vibrators 10 and is made of an insulating material that has an absorbent property and that has a vibration damping property to remove unnecessary weak vibrations from a vibration component of the vibrator 10 so as to prevent the ultrasound waves from the vibrator 10 in the downward direction Z2 from returning to an inside of the vibrator 10 again. The support portion 11A is mainly made of, for example, a urethane resin, an epoxy resin, a silicone resin, or the like. An electrode 16 connected to each of the ground electrode 10C and the signal electrode 10B is provided in the support portion 11A.

The connection portion 11B is made of an insulating material and is mainly made of, for example, a urethane resin, an epoxy resin, a silicone resin, or the like.

In the present embodiment, the respective materials and the like of the support portion 11A, the connection portion 11B, and the third acoustic matching layer 12C constituting a part of a range of the acoustic matching portion 12 in the up-down direction Z are each adjusted so as to have the same thermal conductivity and the same acoustic impedance (substantially the same with tolerable differences). It is preferable that the thermal conductivity of each of the support portion 11A, the connection portion 11B, and the third acoustic matching layer 12C is higher than the thermal conductivity of the acoustic lens 13.

As described above, the support portion 11A, the connection portion 11B, and the third acoustic matching layer 12C have the same thermal conductivity and the same acoustic impedance, so that heat generated in the vicinity of a boundary between the acoustic lens 13 and the acoustic matching portion 12 is more easily transferred to the connection portion 11B having a thermal conductivity higher than the thermal conductivity of the acoustic lens 13. Then, the heat transferred to the connection portion 11B is transferred to the support portion 11A which occupies the majority of the volume in the unit U. Therefore, the safety with respect to the subject under examination can be enhanced by reducing the heat transferred to the acoustic lens 13. In addition, since the connection portion 11B and the acoustic lens 13 are in contact with each other, the above-described heat moved to the acoustic lens 13 can be dissipated to the connection portion 11B and the support portion 11A via a contact surface between the acoustic lens 13 and the connection portion 11B. As a result, the heat transferred to the subject under examination in contact with the acoustic lens 13 can be further reduced.

In addition, the support portion 11A, the connection portion 11B, and the third acoustic matching layer 12C have the same thermal conductivity and the same acoustic impedance, so that, for example, the support portion 11A, the connection portion 11B, and the third acoustic matching layer 12C can be integrally molded using the same material, which makes it possible to reduce the manufacturing cost.

Since required functions (an absorbent property of ultrasound waves) are different between the third acoustic matching layer 12C and the support portion 11A, the acoustic impedances are generally different from each other. The fact that the acoustic impedances are different from each other means that the material compositions are different between the third acoustic matching layer 12C and the support portion 11A. As a result, the thermal conductivity is also generally different between the third acoustic matching layer 12C and the support portion 11A.

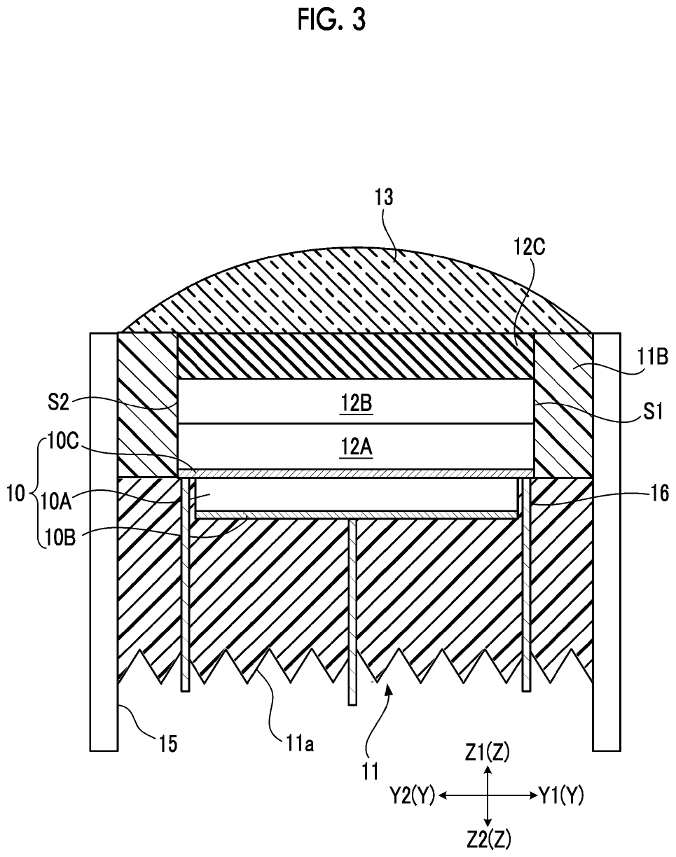

In the present embodiment, by configuring the third acoustic matching layer 12C, the support portion 11A, and the connection portion 11B with, for example, the same material and the same composition, the third acoustic matching layer 12C, the support portion 11A, and the connection portion 11B are made to have the same acoustic impedance and the same thermal conductivity. The acoustic impedances of the third acoustic matching layer 12C, of the support portion 11A, and of the connection portion 11B are designed to, for example, satisfy conditions required for the acoustic matching portion 12. In this case, there is a concern that the support portion 11A may not ensure the sufficient absorbent property of ultrasound waves. However, for the support portion 11A, it is possible to enhance the absorbent property of ultrasound waves, for example, by adjusting its thickness or the like in the up-down direction Z or by devising its shape as in a modification example (FIG. 3), which will be described below. That is, even in a case where the third acoustic matching layer 12C and the support portion 11A are made to have the same acoustic impedance, it is possible to obtain ultrasound images that are practically acceptable without significant problems.

It is also possible to design the acoustic impedances of the third acoustic matching layer 12C, the support portion 11A, and the connection portion 11B to satisfy, for example, a condition required for the support portion 11A. In this case, the absorbent property of ultrasound waves is enhanced in the acoustic matching portion 12. However, in some cases, the advantage may lie in employing high-performance driving for the vibrator 10 rather than focusing on the efficiency of transmission and reception of ultrasound waves. Employing high-performance driving for the vibrator 10 tends to increase the amount of heat generation. However, the third acoustic matching layer 12C, the support portion 11A, and the connection portion 11B have the same thermal conductivity, so that it is possible to restrain heat from being transferred to the subject under examination.

In this way, by making the third acoustic matching layer 12C, the support portion 11A, and the connection portion 11B have the same acoustic impedance and the same thermal conductivity, it is possible to enhance safety while satisfying the performance required for the ultrasound diagnostic apparatus.

It is preferable that a thickness D3 of the connection portion 11B in the front-rear direction Y is made equal to or greater than an average value of an average thickness of the acoustic lens 13 in the up-down direction Z and a thickness of the third acoustic matching layer 12C, which is in contact with the acoustic lens 13, in the up-down direction Z and is made equal to or less than a size that does not cause an influence on a thickness of the ultrasound probe 100 in the front-rear direction due to a thickness influence of the connection portion 11B. By employing such a configuration, the heat dissipation performance through the connection portion 11B and the support portion 11A can be sufficiently ensured, constraint conditions on the ultrasound probe 100 as a medical device can be satisfied, and the manufacturing can be easily performed.

Further, it is preferable that the thickness D3 is greater than a thickness D2 of the third acoustic matching layer 12C in the up-down direction Z. With such a relationship, heat can be efficiently transferred from the third acoustic matching layer 12C to the support portion 11A, and the heat dissipation performance can be further enhanced.

In addition, it is preferable that a thickness D1 of the support portion 11A in the up-down direction Z is at least a thickness such that an acoustic attenuation amount is 40 dB or more in a case where the incident ultrasound wave propagates twice the thickness D1. By employing such a configuration, it is possible to enhance the absorbent property of ultrasound waves of the support portion 11A.

In the ultrasound probe 100, the connection portion 11B is in contact with the entire first end surface S1 and the entire second end surface S2, but various contact forms between the connection portion 11B and the acoustic matching portion 12 can be employed. For example, the connection portion 11B may be in contact with the end surfaces of the third acoustic matching layer 12C and may not be in contact with the end surfaces of the first acoustic matching layer 12A and of the second acoustic matching layer 12B, in the first end surface S1 and the second end surface S2.

Further, the ultrasound probe 100 has a configuration in which the connection portion 11B is provided between the first end surface S1 and the case 15 and between the second end surface S2 and the case 15, but the heat dissipation performance through the third acoustic matching layer 12C, the support portion 11A, and the connection portion 11B can be ensured even in a case where the connection portion 11B between the first end surface S1 and the case 15 is omitted or the connection portion 11B between the second end surface S2 and the case 15 is omitted.

In addition, in the acoustic matching portion 12, a configuration may be employed in which one or both of the first acoustic matching layer 12A and the second acoustic matching layer 12B are made of the same material as that of the third acoustic matching layer 12C. In such a case, the heat dissipation performance can be further enhanced.

FIG. 3 is a view showing a first modification example of the ultrasound probe 100 and is a schematic cross-sectional view corresponding to FIG. 2. The ultrasound probe 100 shown in FIG. 3 has the same configuration as that of FIG. 2 except that the shape of an opposite end part (lower end part) of the support portion 11A from the vibrator 10 side is different.

In this modification example, the shape of the lower end part of the support portion 11A is a shape that allows for scattering of the ultrasound waves radiated from the vibrator 10 to a lower end part side. Specifically, on a lower end surface 11a of the support portion 11A, a plurality of protruding portions having a triangular cross-section and protruding in the downward direction Z2 are arranged in the front-rear direction Y. The cross-sectional shape of the protruding portion is not limited to a triangle and may be a polygonal shape having a three or more sides.

According to the modification example shown in FIG. 3, it is possible to suppress the backward propagation of ultrasound waves radiated from the vibrator 10 to the lower end part side of the support portion 11A toward the vibrator 10 side by using the shape of the lower end part thereof. Therefore, even in a case where the acoustic impedance of the third acoustic matching layer 12C of the acoustic matching portion 12 is lowered (that is, the acoustic impedance of the support portion 11A is lowered) in order to efficiently make the ultrasound wave incident on the subject under examination, the sensitivity or the resolution of the image that can be acquired by the ultrasound probe 100 can be increased.

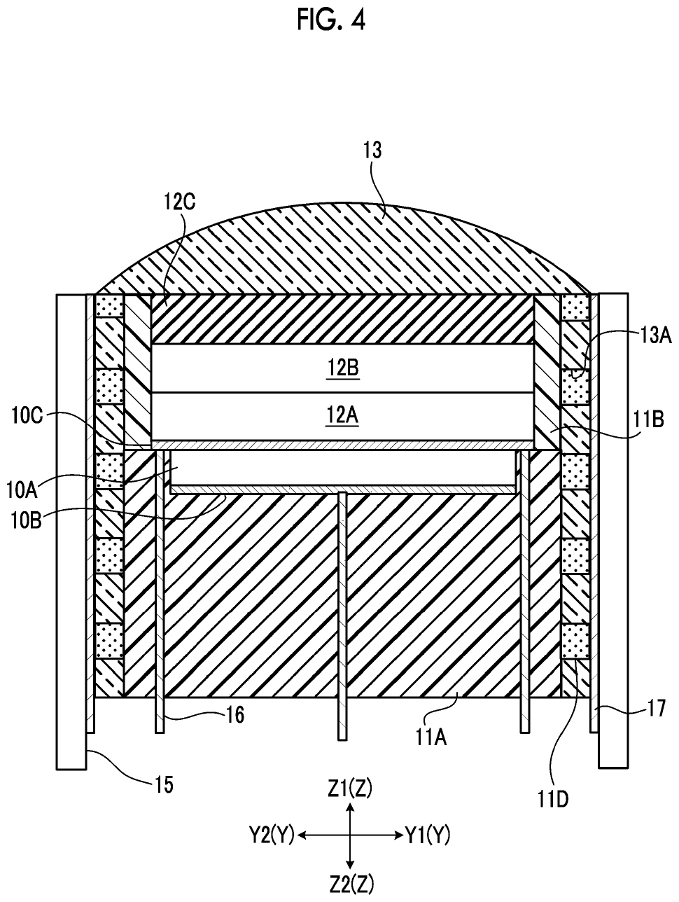

FIG. 4 is a view showing a second modification example of the ultrasound probe 100 and is a schematic cross-sectional view corresponding to FIG. 2. The ultrasound probe 100 shown in FIG. 4 has the same configuration as that shown in FIG. 2 except that a space between the case 15 and the functional part 11 is filled with a support portion that is integrally formed with the acoustic lens 13 and a part of the acoustic lens 13 holds each unit U, that a plurality of through-holes 13A penetrating in the front-rear direction Y are provided in the support portion which is located between the case 15 and the functional part 11 and a filling layer 11D made of a material having a thermal conductivity higher than the thermal conductivity of the acoustic lens 13 is provided in the through-hole 13A, and that a copper plate 17 is added.

The copper plate 17 has a thickness direction that coincides with the front-rear direction Y and is provided between the case 15 and the support portion composed of a part of the acoustic lens 13. The copper plate 17 constitutes a heat dissipation member that is disposed to face the end surfaces of the support portion 11A and of the connection portion 11B in the front-rear direction Y. The copper plate 17 may be made of a metal other than copper or the like.

The filling layer 11D is made of, for example, the same material as that of the support portion 11A or the connection portion 11B. In the filling layer 11D, one end surface in the front-rear direction Y is in contact with the copper plate 17, and the other end surface in the front-rear direction Y is in contact with the support portion 11A and the connection portion 11B.

In the ultrasound probe 100 shown in FIG. 4, since the functional part 11 and the copper plate 17 having a high thermal conductivity are connected to each other through the filling layer 11D, heat generated in the vicinity of the boundary between the acoustic lens 13 and the acoustic matching portion 12 can also be dissipated to the copper plate 17, and the heat dissipation performance can be further enhanced.

The number of the filling layers 11D to be installed (or an installation area) may be changed between a region that overlaps the connection portion 11B when viewed in the front-rear direction Y and a region that overlaps the support portion 11A when viewed in the front-rear direction Y. That is, the contact area between the connection portion 11B and the filling layer 11D and the contact area between the support portion 11A and the filling layer 11D may be different from each other.

For example, in a case where a larger number of the filling layers 11D are provided in the region that overlaps the connection portion 11B when viewed in the front-rear direction Y than in the region that overlaps the support portion 11A when viewed in the front-rear direction Y, it is possible to more efficiently dissipate the heat. A configuration may be employed in which the filling layer 11D is not provided in any one of the region that overlaps the support portion 11A when viewed in the front-rear direction Y or the region that overlaps the connection portion 11B when viewed in the front-rear direction Y.

At least the following matters are described in the present specification.

(1)
An ultrasound probe comprising:
   at least one vibrator having a first surface which is located on a side on which the vibrator transmits and receives an ultrasound wave and a second surface opposite to the first surface;
   a support portion that supports the second surface;
   an acoustic lens that is disposed on a side opposite to a support portion side with respect to the vibrator;
   an acoustic matching portion that is disposed between the vibrator and the acoustic lens; and
   a connection portion that connects a first range and the support portion to each other, the first range being at least a partial range of the acoustic matching portion in a first direction from the acoustic lens toward the vibrator, in which the support portion, the connection portion, and the first range have the same thermal conductivity and the same acoustic impedance.

According to (1), heat generated in the vicinity of the boundary between the acoustic matching portion and the acoustic lens can be dissipated from the first range of the acoustic matching portion to the support portion via the connection portion. As a result, it is possible to reduce heat transferred to the subject under examination in contact with the acoustic lens, and it is possible to reduce constraints on a driving method of the vibrator. For example, it is also possible to increase a driving voltage of the vibrator, and it is possible to improve the sensitivity or the image quality of the ultrasound image generated by using the ultrasound probe.

(2)
The ultrasound probe according to (1),
   in which the first range, the connection portion, and the support portion are made of the same material.

According to (2), the first range, the connection portion, and the support portion can be integrally formed, so that the manufacturing can be easily performed. In addition, it is possible to easily make the first range, the connection portion, and the support portion have the same thermal conductivity and the same acoustic impedance.

(3)
The ultrasound probe according to (1) or (2),
   in which the first range includes a contact surface which is contact with the acoustic lens.

According to (3), the heat of the contact surface between the acoustic matching portion and the acoustic lens, which may generate more heat, can be dissipated to the support portion, so that the heat transferred to the subject under examination in contact with the acoustic lens can be effectively reduced.

(4)
The ultrasound probe according to any one of (1) to (3),
   in which the at least one vibrator includes a plurality of vibrators,
   the plurality of vibrators are arranged in a second direction along a plane intersecting the first direction, and
   a length of the connection portion in a third direction which is a direction extending along the plane and intersecting the second direction is greater than a length of the first range in the first direction.

According to (4), heat generated in the vicinity of the boundary between the acoustic matching portion and the acoustic lens can be more efficiently dissipated to the support portion.

(5)
The ultrasound probe according to any one of (1) to (4),
   in which the connection portion is in contact with the acoustic lens.

According to (5), heat generated in the vicinity of the boundary between the acoustic matching portion and the acoustic lens can be more efficiently dissipated to the support portion.

(6)
The ultrasound probe according to any one of (1) to (5),
   in which the acoustic matching portion includes a plurality of layers laminated in the first direction, and
   the first range is a layer of the acoustic matching portion closest to the acoustic lens among the plurality of layers.

According to (6), heat generated in the vicinity of the boundary between the acoustic matching portion and the acoustic lens can be more efficiently dissipated to the support portion. In addition, it is possible to perform good matching of the acoustic impedances between the subject under examination and the vibrator, which makes it possible to efficiently perform the transmission and reception of ultrasound waves.

(7)
The ultrasound probe according to any one of (1) to (6),
   in which the at least one vibrator includes a plurality of vibrators,
   the plurality of vibrators are arranged in a second direction along a plane intersecting the first direction,
   the ultrasound probe further comprises a heat dissipation member that is disposed to face end surfaces of the support portion and of the connection portion in a third direction which is a direction extending along the plane and intersecting the second direction is defined as a third direction, and
at least one of the support portion or the connection portion is connected to the heat dissipation member.

According to (7), heat generated in the vicinity of the boundary between the acoustic matching portion and the acoustic lens can also be dissipated to the heat dissipation member. Therefore, constraints on the thermal conductivity or the acoustic impedance in the support portion, the connection portion, and the first range can be relaxed, and the manufacturing can be easily performed.

(8)
The ultrasound probe according to any one of (1) to (7),
   in which the support portion has an end part on a side opposite to the vibrator;
   the end part of the support portion has a shape that allows an ultrasound wave radiated from the vibrator to the side of the end part to be scattered.

According to (8), it is possible to suppress the backward propagation of ultrasound waves radiated from the vibrator to the end part side of the support portion toward the vibrator side by using the shape of the end part thereof. Therefore, even in a case where the acoustic impedance of the first range of the acoustic matching portion is lowered in order to efficiently make the ultrasound wave incident on the subject under examination, the sensitivity or the resolution of the image that can be acquired by the ultrasound probe can be increased.

(9)
An ultrasound diagnostic apparatus comprising:
   the ultrasound probe according to any one of (1) to (8).

### EXPLANATION OF REFERENCES

S1: first end surface
S2: second end surface
10A: piezoelectric body
10B: signal electrode
10C: ground electrode
10: vibrator
11A: support portion
11B: connection portion
11D: filling layer
11a: lower end surface
11: functional part
12A: first acoustic matching layer
12B: second acoustic matching layer
12C: third acoustic matching layer
12: acoustic matching portion
13A: through-hole
13: acoustic lens
14: separation layer
15: case
16: electrode
17: copper plate
100: ultrasound probe

What is claimed is:

1. An ultrasound probe comprising:
at least one vibrator having a first surface which is located on a side on which the vibrator transmits and receives an ultrasound wave and a second surface opposite to the first surface;
a support portion that supports the second surface;
an acoustic lens that is disposed on a side opposite to a support portion side with respect to the vibrator;
an acoustic matching portion that is disposed between the vibrator and the acoustic lens; and
a connection portion that connects a first range and the support portion to each other, the first range being at least a partial range of the acoustic matching portion in a first direction from the acoustic lens toward the vibrator,
wherein the support portion, the connection portion, and the first range have the same thermal conductivity and the same acoustic impedance.

2. The ultrasound probe according to claim 1,
wherein the first range, the connection portion, and the support portion are made of the same material.

3. The ultrasound probe according to claim 2,
wherein the first range includes a contact surface which is in contact with the acoustic lens.

4. The ultrasound probe according to claim 1,
wherein the at least one vibrator includes a plurality of vibrators,
the plurality of vibrators are arranged in a second direction along a plane intersecting the first direction, and
a length of the connection portion in a third direction which is a direction extending along the plane and intersecting the second direction is greater than a length of the first range in the first direction.

5. The ultrasound probe according to claim 2,
wherein the at least one vibrator includes a plurality of vibrators,
the plurality of vibrators are arranged in a second direction along a plane intersecting the first direction, and
a length of the connection portion in a third direction which is a direction extending along the plane and intersecting the second direction is greater than a length of the first range in the first direction.

6. The ultrasound probe according to claim 3,
wherein the at least one vibrator includes a plurality of vibrators,
the plurality of vibrators are arranged in a second direction along a plane intersecting the first direction, and
a length of the connection portion in a third direction which is a direction extending along the plane and intersecting the second direction is greater than a length of the first range in the first direction.

7. The ultrasound probe according to claim 1,
wherein the connection portion is in contact with the acoustic lens.

8. The ultrasound probe according to claim 2,
wherein the connection portion is in contact with the acoustic lens.

9. The ultrasound probe according to claim 3,
wherein the connection portion is in contact with the acoustic lens.

10. The ultrasound probe according to claim 1,
wherein the acoustic matching portion includes a plurality of layers laminated in the first direction, and
the first range is a layer of the acoustic matching portion closest to the acoustic lens among the plurality of layers.

11. The ultrasound probe according to claim 2,
wherein the acoustic matching portion includes a plurality of layers laminated in the first direction, and
the first range is a layer of the acoustic matching portion closest to the acoustic lens among the plurality of layers.

12. The ultrasound probe according to claim 3,
wherein the acoustic matching portion includes a plurality of layers laminated in the first direction, and
the first range is a layer of the acoustic matching portion closest to the acoustic lens among the plurality of layers.

13. The ultrasound probe according to claim 1,
wherein the at least one vibrator includes a plurality of vibrators,
the plurality of vibrators are arranged in a second direction along a plane intersecting the first direction,
the ultrasound probe further comprises a heat dissipation member that is disposed to face end surfaces of the support portion and of the connection portion in a third direction which is a direction extending along the plane and intersecting the second direction is defined as a third direction, and
at least one of the support portion or the connection portion is connected to the heat dissipation member.

14. The ultrasound probe according to claim 2,
wherein the at least one vibrator includes a plurality of vibrators,
the plurality of vibrators are arranged in a second direction along a plane intersecting the first direction,
the ultrasound probe further comprises a heat dissipation member that is disposed to face end surfaces of the support portion and of the connection portion in a third direction which is a direction extending along the plane and intersecting the second direction is defined as a third direction, and
at least one of the support portion or the connection portion is connected to the heat dissipation member.

15. The ultrasound probe according to claim 3,
wherein the at least one vibrator includes a plurality of vibrators,
the plurality of vibrators are arranged in a second direction along a plane intersecting the first direction,
the ultrasound probe further comprises a heat dissipation member that is disposed to face end surfaces of the support portion and of the connection portion in a third direction which is a direction extending along the plane and intersecting the second direction is defined as a third direction, and
at least one of the support portion or the connection portion is connected to the heat dissipation member.

16. The ultrasound probe according to claim 1,
wherein the support portion has an end part on a side opposite to the vibrator;
the end part of the support portion has a shape that allows an ultrasound wave radiated from the vibrator to the side of the end part to be scattered.

17. The ultrasound probe according to claim 2,
wherein the support portion has an end part on a side opposite to the vibrator;
the end part of the support portion has a shape that allows an ultrasound wave radiated from the vibrator to the side of the end part to be scattered.

18. The ultrasound probe according to claim 3,
wherein the support portion has an end part on a side opposite to the vibrator;
the end part of the support portion has a shape that allows an ultrasound wave radiated from the vibrator to the side of the end part to be scattered.

19. An ultrasound diagnostic apparatus comprising:
the ultrasound probe according to claim 1.
